(19) United States
(12) Patent Application Publication
(10) Pub. No.: US 2004/0138820 A1
(43) Pub. Date: Jul. 15, 2004

(54) Method to control body weight

(76) Inventors: Deborah A. Morris, Venedocia, OH (US); W. Calvin Morris, Venedocia, OH (US)

Correspondence Address: Michael Paolucci, Suite 425, 120 East Fourth Street, Cincinnati, OH, 45202, US

(21) Appl. No.: 10/340,108

(22) Filed: Jan. 10, 2003

(51) Int. Cl. G06F 19/00; G01N 33/48; G01N 33/50

(52) U.S. Cl. 702/19

(57) ABSTRACT

A method for controlling body weight. The method is based on assigning a Amples value to food items based on the mathematical calculation: Amples value=(E/P)/S, where E is the commonly expressed kilocalorie content of the food item; P is the propensity of the food item to elicit Passive Over Consumption; and S is the ability of the food item to create the feeling of satisfaction. Values are assigned to all food items based on observation, experience, case studies and education. Values can be changed for each individual based on their own experiences. Amples are tabulated in any easy to read reference form. The serving sizes upon which the calculations are based on readily estimable amounts using everyday spoons, bowls, plates, and baking dishes with values assigned based on an across-the-board average of all food items.

### REFERENCE TO RELATED APPLICATIONS

This is the first application for this invention.

### REFERENCE TO MICROFICHE APPENDIX

None.

### FIELD OF THE INVENTION

A method of maintaining current weight and reducing current weight.

### BACKGROUND OF INVENTION

This invention relates to the area of eating, dieting, weight control, nutritional education and the behavioral aspects of eating.

Obesity is epidemic in the Industrialized World today. The conditions of obesity and being overweight affect over 61% of American adults according to government studies, and they cost the economy billions of dollars a year in healthcare costs and lost productivity due to illnesses caused or exacerbated by exogenous fat. In response, a weight loss industry has grown into the tens of billions of dollars a year, and hundreds of plans and methods of weight control have arisen.

In spite of this growth, studies have shown that the failure rate, or recalcitrance rate, for obesity is high regardless of which method of dieting is used. From these studies it has been proven that over 90% of dieters regain their weight in two to five years. Only 2% manage a sustained weight loss for more than twenty four (24) months.

It is certain that no matter how well intended and scientifically based a weight loss plan, long term sustained weight loss will be elusive if the diet participant is unable or unwilling to maintain the program's disciplines. A program that forbids carbohydrates, often bread or pasta, breaks down when the craving for bread or pasta overwhelms the participant's desire to remain compliant. The same is true for programs that limit fat intake, or any other major source of nutrition.

More balanced programs, those that allow foods in moderation, in practice fail because they steer their participants to eat lower calorie meals rather than answering food cravings. Balanced programs that involve calorie counting, or calorie plus some factor (calories divided by some number to simplify adding) require careful execution to be successful. Tedious measuring and complex food deconstruction to determine calorie content requires frequent references to calorie or factored calorie guides. This added layer of complexity increases recalcitrance rates. Participants in these programs must calculate mathematically what they are permitted to eat at the expense of what they are craving. These mathematical calculations can be quite complex, particularly when multi-ingredient food deconstruction is required.

Taken together, these problems compound to defeat long term weight control. In order to account for the problems of conventional weight loss/control programs, the inventors have developed a process that emphasizes long term weight management from its onset. Complexity and Complexity and unanswered cravings are substantial issues common to prior art weight loss programs that contribute to the high recalcitrance rate referenced above.

To lose weight a person must consume less food energy than needed to maintain their metabolism and fuel their activities. In overweight or obese individuals, this limiting of food intake runs counter to years of learned eating and activity habits, and is believed to conflict with certain elements of their genetic background that has led them to be overweight or obese. Modifying the person's dietary intake sets off various physiologic responses that frequently lead to strong and often uncontrollable food cravings. It is these cravings that make long term weight loss an almost universally frustrating experience.

The inventors have developed a methodology of modifying food energy intake to achieve the goal of reducing body weight or maintaining an already achieved weight loss by specifically confronting and satisfying food cravings. This has been determined to greatly enhance the chances of sustaining weight loss over time. In addition to unmet food cravings, another important factor is the phenomena of Passive Over Consumption. Studies have found that people have a tendency to eat more than they intend without realizing that they are doing so. This is referenced by the inventors as the Passive Over Consumption element.

Prior art weight loss and weight control programs focus on restriction of specific food groups or restriction of all food groups. The aforementioned prior art programs produce weight loss by decreasing average daily food energy intake, but achieve the goals at the expense of feeling satisfied from eating. The weight loss or control requires denial of food cravings and thus involves a significant sacrifice in one's eating habits. The failure to deal with food cravings of the dieting individual is a significant disadvantage in these prior art plans; requires that the dieter sacrifice their favorite foods; and does so at the expense of feeling satisfied.

The prior art plans vary in complexity, but most require close consideration of the specific contents of foods so as to exclude or include specific food groups; e.g. fats or carbohydrates. Many of the prior art plans, while more balanced in their approach to dieting, still require intensive tracking of portions; reference to voluminous charts listing the calorie (or other units) content of various foods; or other aides; and often complex mathematical calculations to ascertain the caloric content of a single serving of a multi-ingredient dish. These prior art programs generally fail by noncompliance within a few weeks of inception due to their complexity; due to the failure to satisfy the participants; and due to other similar problems with the amount of sacrifice necessary to achieve the goals. While some of the prior art programs may result in significant weight loss, the scientific and medical literature show that the failure, or recalcitrance rate is extremely high: greater than 90% within three (3) to five (5) years. The pattern will typically consist of an initial period of weight loss followed by a period where the unwanted weight returns during which it is also common for the user's weight to actually increase over the starting weight, due to the failure of conventional programs to foster long term compliance.

An important consideration of the present invention is designing a method that does not require an unreasonable sacrifice in the amount of and types of foods that a person normally consumes. While a reduction in calorie consumption will follow from a change in eating habits, such is an ancillary affect of the invention. The goal is to change dietary habits so that a healthier lifestyle results without the dramatic sacrifice of the prior art methods and systems.

The present invention recognizes that long term compliance and weight control hinge on the regular and ongoing satisfaction of food cravings. The inventors have identified this tendency of foods to create the feeling of Satisfaction and have assigned values to all food items based on said tendency. This is referenced by the inventors as the Satisfaction (S) element.

Programs that are based on restrictive dieting fail to address the person's need for satisfaction, confounding their longer term success beyond an initial rapid weight loss phase. These programs rely on restrictions and guilt, instead of teaching enlightened eating behaviors that emphasize the satisfaction of food cravings to help lose weight and make long term weight control an attainable goal.

The present invention also recognizes that mere calorie and factored calorie counting, with their continual associated, and often complex, calculations that are necessary to track an ordinary human diet, are cumbersome, difficult, and lead to poor long term compliance. To address that shortcoming of the prior art, the present invention provides a plan that is easy to follow without being intrusive and without requiring ongoing complicated mathematical calculations. A fundamental principle of the present invention is to encourage the satisfaction of cravings while meeting a person's nutritional needs within an easily learned framework that encourages meaningful weight control over the long term.

The present invention addresses the shortcomings of the prior art in that it is easily learned, provides a solid structure, and is non-restrictive. To achieve these goals, a person may eat any food they crave. Simply put, no foods are prohibited. It is an important principle of the present invention that the method does not impose unreasonable and therefore unsustainable sacrifice in the amount and types of foods that a person is guided to consume. A fundamental principle of the present invention is to change dietary habits so that weight loss with its associated health benefits, is achieved in a way that encourages long term compliance by limiting the dietary restrictions imposed on the user and by limiting the user's perceived sacrifice.

The inventors have also determined that one reason that the recalcitrance rate is so high is due to the difficulty in tracking the diet. One of the reasons tracking food intake is so difficult is the complexity used in portioning the food that is consumed. The present invention employs several methods for making the portioning of food simple and easy. The methods involve assigning Amples values to all food items across the board, regardless of type of food, based on the amount consumed using the measuring tools of everyday flatware and plates such as: a teaspoon, a tablespoon, a flatware serving spoon, and a buffet spoon, typically found in restaurants. In addition, bowls, plates and baking dishes are given Ample values. The Amples values assigned are premised on a broad average, considering the food consumed in the user's diet, and rely upon the user maintaining the balance of those foods for success.

The present invention also alternatively quantitatively assigns Ample values to specific food items, summarized in simple, user friendly tables, by identifying and quantifying three characteristics of each food item: food energy expressed as the calorie content (E); the tendency of a food item to lead to Passive Over Consumption (P); and the tendency of the food item to create a feeling of Satisfaction (S). The present invention uses these quantified elements in an easy to follow program that allows participants to either lose or control their weight. The present invention thus uses each of the characteristics for every food item, Calorie, Passive Over Consumption, and Satisfaction, to produce a new characteristic number, its Amples Value, that, once rounded to the nearest multiple of five, allows a participant to maintain sustained weight control or weight loss. The Amples value may be used to monitor each item consumed, or it may be used within the framework of a broad, across the board average based on the size of portion consumed, regardless of the content of the food item consumed.

A prior art method of dieting is disclosed in U.S. Pat. No. 6,040,531, to Miller-Kovach, et al. wherein a diet is a function based on measured calories, total fat, and dietary fiber for serving sizes specified in readily estimable units. This prior art is distinguishable in that it relies on factors not included in the present invention, total fat and dietary fiber, and does not identify, account for or appreciate the importance of the characteristics of passive over consumption, and ability to create lasting satisfaction of food items.

Other prior art exists for methods of dieting exist, including U.S. Pat. No. 5,640,774; U.S. Pat. No. 4,606,555; U.S. Pat. No. 5,832,446; U.S. Pat. No. 6,296,488; and U.S. Pat. No. 6,083,006. Although each of these patents provide a method for controlling diet, none use the factors or method embodied in the present invention, as hereinafter set forth.

### OBJECTIVES OF THE INVENTION

One objective of the invention is to create a method of controlling body weight by factoring in a user's need to be satisfied.

It is a further objective of this invention to allow substantial but controlled weight loss initially while laying the ground work for sustained weight control using minor modifications of the skills used to accomplish the initial weight loss.

Yet another objective of this invention is to provide a method of weight control that allows an individual to maintain a current weight once the objectives have been reached such that the invention is more than a weight loss program, it is a weight control program through education as well as through changed eating habits.

It is another objective of the invention to provide an easy to follow method of weight control by allowing participants to follow the method wherever, whenever, and under any circumstance without having to make complicated calculations; refer to detailed charts; or make intensive calculations to continue with the program.

Yet another objective of the invention is to create an easy to learn method of dieting that accounts for nutritional needs, satisfaction, and psychology of an individual.

It is a further objective of the invention to create a method of dieting that accounts for the tendency of food to create a feeling of Satisfaction.

Yet, another objective of the invention is to create a method of dieting based on the tendency of food to cause Passive Over Consumption.

It is another objective of the invention is to provide an easy to follow method whereby a participant can either control their current weight or lose weight.

It is another objective of the invention is to provide a sustained state of weight control, with an easily learned methodology.

It is yet another objective of the invention to allow the tracking of consumption without the use of special measuring devices, and to allow the tracking consumption by use of ordinary, every day serving units, utensils and dishes, such as flatware teaspoons, flatware tablespoons, flatware serving spoons, bowls, and plates.

Yet another objective of the invention is to create a diet that allows the consumption of all foods.

Yet another objective of the invention is to create a diet that is portable, and in so doing, that is easily moved into any dining situation, private or public.

A further objective of the invention is to provide a dietary control process that accounts for the user's food preferences, that accounts for the ability of the food preferences to create Satisfaction, and that accounts for the tendency of the food to cause Passive Over Consumption.

A further objective of the invention is to account for three (3) factors, food energy expressed as Calories, Passive Over Consumption, and Satisfaction, in one (1) easy to follow number that a user can apply in developing a diet plan.

A further objective of the invention is to remove the need to count calories in controlling weight. This is accomplished by calculating and assigning characteristic values, hereinafter Amples values, for all food items and assembling them into a simple user friendly reference table. Based on these Amples values, an individual can determine the appropriate amount of food to consume. The Amples values for each food item take into consideration the food energy of the food item, expressed as caloric content, but also factors in the tendency of the food item to satisfy; and the tendency of the food item to elicit Passive Over Consumption. In addition, the method may be followed by rounding, up or down, to the nearest multiple of five, in tracking the diet, without varying from the overall dietary plan.

These and other objects of the invention will be apparent to those skilled in this art from the following detailed description of a preferred embodiment of the invention.

### BRIEF SUMMARY OF THE INVENTION

The present invention provides, first, a method for deriving a characteristic value, known as an Amples Value for a food item. The method for deriving an Amples value comprises several steps, the first of which is adopting the accepted kilocalorie value for the particular food item, hereinafter referenced as E. Next, a Passive Over Consumption value is assigned to the food item based upon the propensity of the food item to elicit over consumption, hereinafter referenced as P. The Passive Over Consumption value is assigned based on the inventors experience, education, test results, and case studies. Next, a Satisfaction value is assigned to the food item based on the propensity of the food item to create the feeling of satisfaction, hereinafter referenced as S. The Satisfaction value is assigned based on the inventors experience, education, test results, and case studies. The Amples value for the food item is then calculated based on the formula: Amples Value=(E/P)/S.

In another preferred embodiment of the present invention, the Amples values, A, are measured based on the units used to serve food by using a broad, across the board average of the Amples values of specific food items as calculated above in (E/P)/S. By using common serving units (utensils and dishes) such as a flatware teaspoon, a flatware tablespoon, an ordinary bowl, or an ordinary plate, an Amples Value for multi-ingredient items, for example spaghetti and meatballs, is assigned on a per portion basis based on the average of all foods that can fit within that serving unit. Thus, regardless of the food item served with that serving unit, an Amples Value is assigned per portion for the amount served based on a broad, across the board average. The units utilized in the present invention are a flatware teaspoon, a heaping flatware teaspoon, a flatware tablespoon, a heaping flatware tablespoon, a flatware serving spoon, a buffet spoon, an ordinary bowl, and an ordinary plate, although other common serving units or dishes may be used or substituted without departing from the principles of the present invention.

In a preferred embodiment of the present invention the Passive Over Consumption value, P, is assigned a value ranging from 0.1 to 2.0. This value for a food item is the result of the experience, education, test results, and case studies of the inventors. In the preferred embodiment of the present invention the Satisfaction value, S, is assigned a value ranging from 0.1 to 4.0. This value for a food item is also the result of the experience, education, test results, and case studies of the inventors.

In a preferred embodiment of the present invention the method of calculating Amples Values, set forth above, is applied to create a diet plan. A Target Weight, T, is chosen by the user of the program. Next, the Target Weight, T, is multiplied by a factor of nine (9) to obtain an Amples Budget, AB, that is the maximum number of Amples that the user may consume in a one day period. Next, the Amples Values for the food items consumed for the day are added and tracked during a one day period the sum thereof being referenced as the Total Amples, TA. By tracking the Total Amples, TA, consumed, the user can insure that the Amples Budget is not exceeded.

In another preferred embodiment of the present invention, the method of adding and tracking Total Amples, TA, and not exceeding the Amples Budget, AB, is modified according to the unique characteristics of the user. Where particular users may vary in the affect food items have on either feelings of Satisfaction, S, or on tendency for Passive Over Consumption, P, the principles of the present invention allow the target weight, T, to be modified to avoid said cravings.

### BRIEF DESCRIPTION OF THE DRAWING

FIG. 1 is a quick reference table with Amples values assigned to numerous food items, including, breads, meats, dairy, potatoes, seafood, and hot or cold casseroles.

FIG. 2 is a quick reference table with Amples values assigned to numerous food items including, Veggies, Fruits, Fast Food, and Ethnic Dining.

FIG. 3 is a table for Dairy Food items further subdivided into categories with columns across the top representing the food item's characteristic quantities, specifically the Kilocalorie, E, the propensity for the food item to elicit Passive Over Consumption, P, and the tendency of the food item to create the feeling of Satisfaction, S. The last two columns of each graph are the resulting Amples Value for each category with the final column being the Amples Value for the food item, rounded to the nearest multiple of five.

FIG. 4 is a table for Meat items further subdivided into categories with columns across the top representing the food item's characteristic quantities, specifically the Kilocalorie, E, the propensity for the food item to elicit Passive Over Consumption, P, and the tendency of the food item to create the feeling of Satisfaction, S. The last two columns of each graph are the resulting Amples Value for each category with the final column being the Amples Value for the food item, rounded to the nearest multiple of five.

FIG. 5 is a table for Potato items further subdivided into categories with columns across the top representing the food item's characteristic quantities, specifically the Kilocalorie, E, the propensity for the food item to elicit Passive Over Consumption, P, and the tendency of the food item to create the feeling of Satisfaction, S. The last two columns of each graph are the resulting Amples Value for each category with the final column being the Amples Value for the food item, rounded to the nearest multiple of five.

FIG. 6 is a graph with assigned Amples values for common serving units, flatware teaspoon, flatware tablespoon, flatware serving spoon, buffet spoon, bowl, plate and baking dish, based on a broad, across the board average of the Amples values for all food items in a user's diet.

### DETAILED DESCRIPTION

Weight loss programs are ubiquitous. They appear everywhere because more than half of all Americans are defined as obese or overweight. Although many types of diet programs are well known and well advertised, their failures are equally well-known. Indeed, the only thing more well-accepted than the notion that Americans are overweight is that the diet programs currently available fail to assist those same Americans cure their overweight condition.

It is well accepted within the field of nutrition that most diet programs do not help correct overweight conditions of users, especially over the long term. The problems are that the users stop the programs because their cravings are not satisfied; the users stop the programs because their need to eat certain foods go unfulfilled; and because the programs do not teach them how to eat better, they only force users to sacrifice the foods they desire. Consequently, the prior art does not account for the need to feel satisfied; does not allow users to eat whatever they want; unrealistically prohibit the consumption of foods; do not account for the cravings that people naturally have for certain foods; are difficult to follow; are difficult to calculate whether the diet is being followed; and generally fail to create a realistic, easy to follow method of weight control.

The present invention provides a method that overcomes the shortcomings of the prior art which lead to a high recalcitrance rate among dieting participants. Instead of tracking calories, fat, carbohydrates, or other such items, the present invention utilizes a newly defined characteristic defined as the Amples value (A) of a food item. Amples values (A) are assigned to every food item in a preferred embodiment of the invention and are used in calculating an across the board, per portion average to every serving unit of food. Once the Amples values (A) are assigned, a participant chooses a target weight (T), creates an Amples Budget (BA), and then more easily stays within that budget by monitoring his consumed Amples (CA) such that they are equal or less than the Amples Budget (AB) allows. Since Amples values are rounded, no complicated, fractional mathematical calculations are necessary and accounting for the number of consumed Amples (CA) is easy.

Amples values (A) are calculated by first determining the number of kilocalories (E) any food item has. This is obtained from either the USDA, Nutrient Data Laboratory, Agricultural Research Service, Nutrititive Value of Foods, Home and Garden Bulleting, HG-72; from the source of the food, such as the restaurant that provides the information pursuant to law; or from other similar accepted within the art sources of kilocalories, hereinafter, E.

Once E is obtained, a number is assigned to each food item based on its tendency to cause Passive Over Consumption (P). Passive Over Consumption is a characteristic of a food item, quantified in the present invention, recognizing the tendency of the food item to be unintentionally overconsumed. While the broad idea of Passive Over Consumption is known in the art, there is no prior art that either quantifies the element, or accounts for the element in a diet. The present invention assigns Passive Over Consumption values (P) to all food items ranging between 0.1 and 2.0, with 0.1 being a food item that exhibits a low propensity for overconsumption, and 2.0 being a food item that exhibits a high propensity for overconsumption. In the present invention, the Passive Over Consumption values (P) are assigned based upon the inventors' experience, education, test results, and case studies.

In addition to assigning values that account for the tendency of a food item to cause Passive Over Consumption, the present invention assigns values based on the tendency of a food item to create the feeling of Satisfaction (S). Similar to Passive Over Consumption, while the literature in the art has recognized that the ability of food to create the feeling of Satisfaction is important in preventing overeating, none of the prior art has quantified Satisfaction values for food items or applied it in creating a diet plan. By assigning a quantified Satisfaction value (S) to food items based on this tendency to create a feeling of Satisfaction, the present invention accounts for this food characteristic. The present invention assigns Satisfaction values (S) to all food items between 0.1 and 4.0, with 0.1 being a food item that exhibits a low tendency to create a feeling of Satisfaction and 4.0 being a food item that exhibits a high tendency to create a feeling of Satisfaction. In the present invention, the Satisfaction values (S) are assigned based upon the inventors' experience, education, test results, and case studies.

With these three (3) characteristics quantified (kilocalories count (E), Passive Over Consumption (P), and Satisfaction (S)), every food item is then assigned its characteristic Amples value (A) by use of the formula Amples Value=(E/P)/S. Amples values (A) are rounded to the nearest multiple of five for ease of calculation.

Once Amples Values (A) are assigned for all of the food items in a user's diet, they can be utilized to arrive at a common representative factor defined as a per portion Amples average (AAVG), regardless of the food being consumed. This allows a user to maintain his current diet and simply monitor consumed Amples (CA) based on the size of the portions consumed, regardless of the actual food item consumed. This is accomplished by defining the serving unit Amples values (SUA) for quantities in ordinary serving units, normal flatware and dishes, regardless of the type of food served with the serving unit. While one preferred embodiment of the present invention defines the serving units Amples values (SUA) for multi-ingredient foods, like casseroles, the same principle can be extended to apply for all foods.

The per portion Amples average (AAVG) is derived by using an across-the-board average of the Amples values (A) of all food items utilizing the (E/P)/S calculation. After calculating the per portion Amples average value (AAVG) of all food items in a user's diet, this Amples average value (AAVG) is distilled into units based on everyday serving units. In this way ordinary utensils and dishes, e.g. a flatware teaspoon, a heaping flatware teaspoon, a flatware tablespoon, a heaping flatware tablespoon, a flatware serving spoon, a buffet spoon, a traditional dinner bowl, a traditional dinner dish or a baking dish, are assigned serving unit Amples values (SUA) based on the average of all foods, regardless of the content of the serving unit.

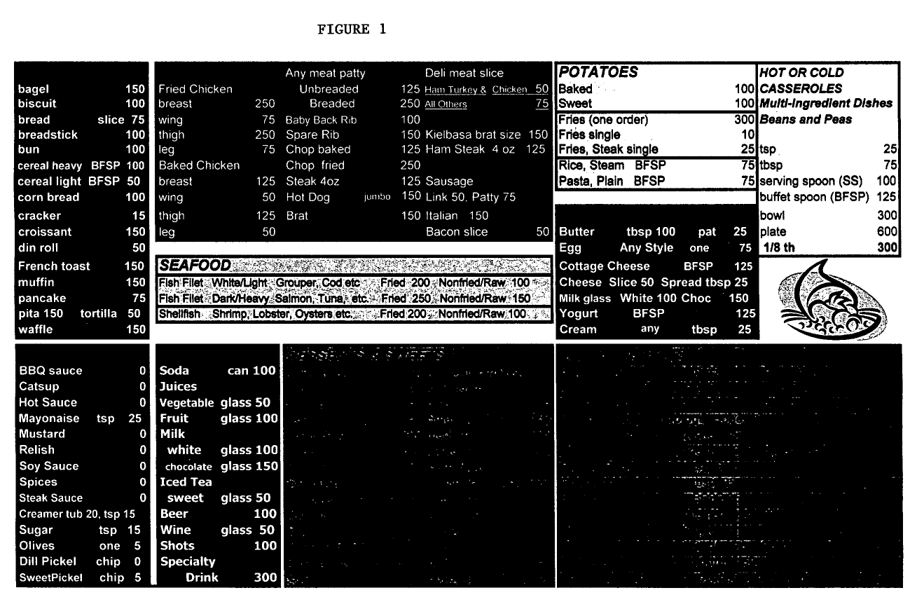

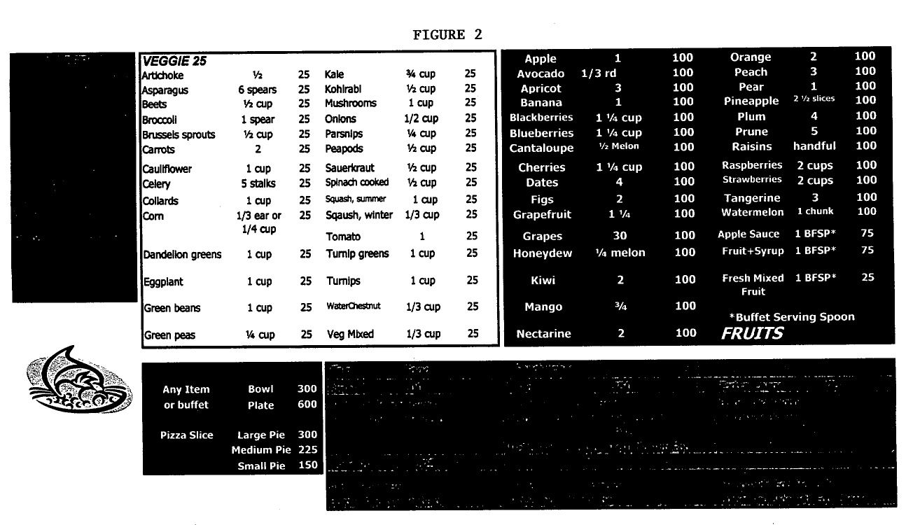

In a preferred embodiment of the invention, the Amples values (A) for common food items are assembled into an easy to read chart shown in FIGS. 1 and 2. With the chart, a participant can easily determine the number of consumed Amples (CA) for all food items eaten in a day and consciously monitor and compare that to his Amples budget (AB). FIG. 1 reflects Amples values for a wide variety of common food items, derived from the calculation accounting for calorie content (E), passive overconsumption (P) and satisfaction (S) characteristics of the food items. FIG. 2 lists Amples values (A) for other common food items and is the reverse side of the chart depicted in FIG. 1, which, printed front and back, laminated and reduced to the size of a credit card provides a convenient pocket reference for users who monitor consumed Amples (CA) by summing the Amples values (A) of the specific food items he/she eats during the day.

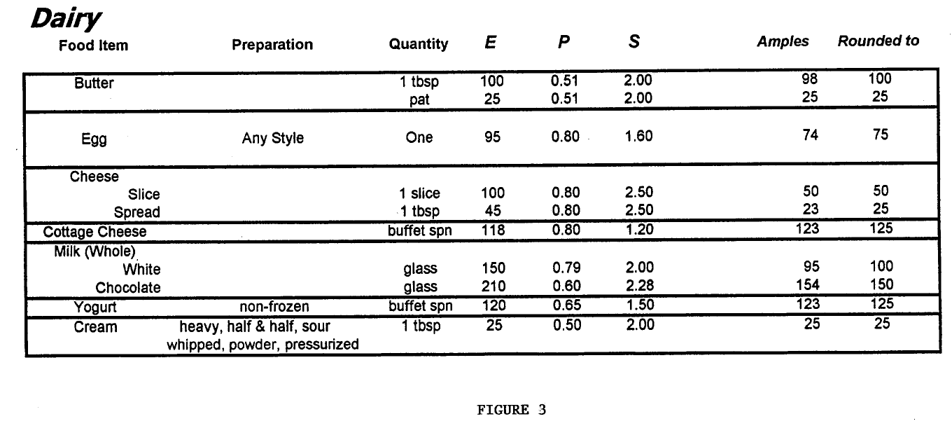

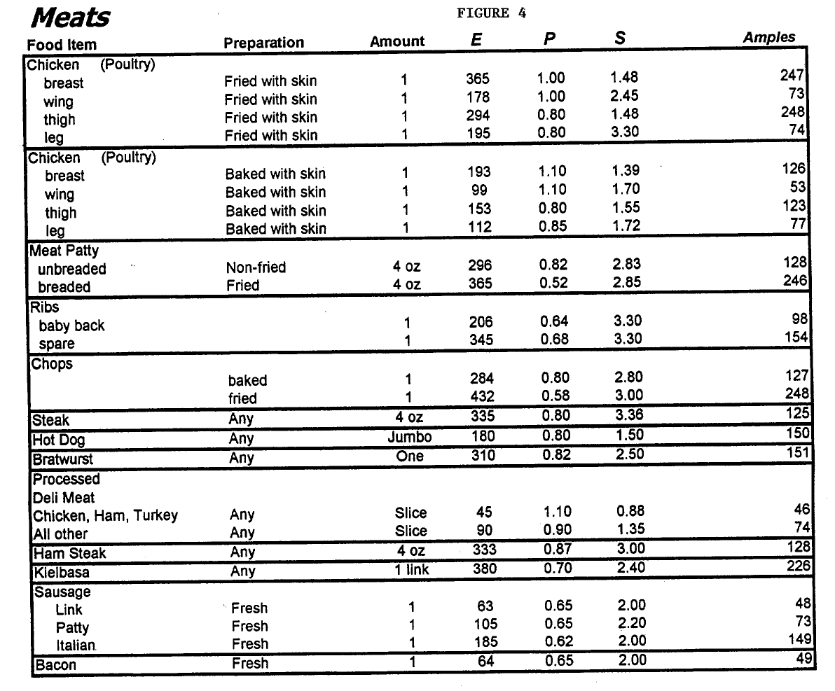

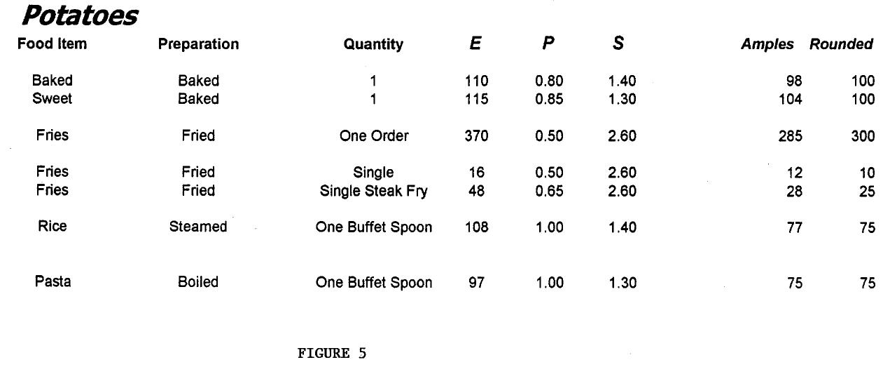

FIGS. 3, 4, and 5 are more detailed charts reflecting common food items divided into subcategories depending on the general classification of the food item. FIGS. 3, 4 and 5 reflect not only the Amples values (A) of each food item, but also the assigned values for Passive Over Consumption (P) and Satisfaction (S). Each chart shows the assignment of Kilocalories, E, Passive Over Consumption, P, and Satisfaction, S. Each also shows that Amples Values are derived from these three (3) elements, and is then rounded to arrive at a final Amples Value (A). FIGS. 3, 4 and 5 reflect typical charts listing Amples values (A) for well defined food groups, but other charts listing different food items, or listing food items organized differently, are specifically contemplated and do not depart from the principles of the present invention.

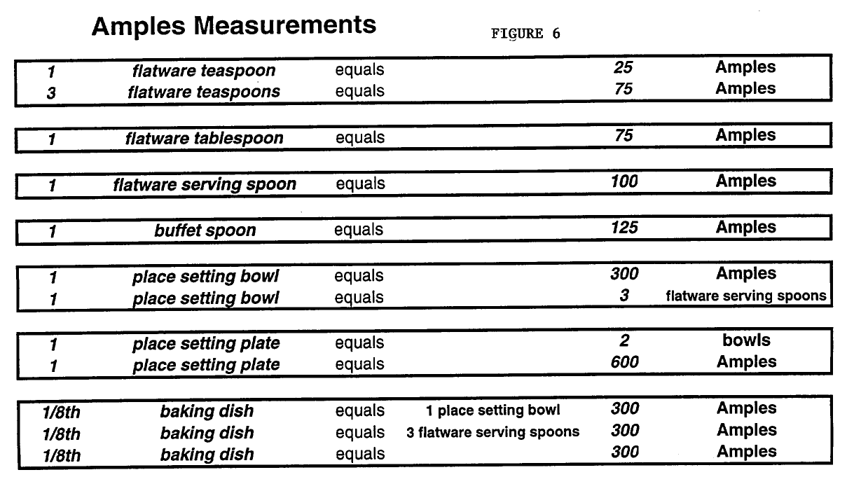

FIG. 6 is a chart listing serving units Amples values (SUA) for common serving utensils and dishes, e.g. flatware teaspoon, flatware tablespoon, buffet spoon, bowl, plate and baking dish. Once Amples Values are assigned, it is necessary to determine how many Amples per day are consumable to either lose weight or to maintain current weight. The serving unit Amples values (SUA) are calculated by taking an across the board average of Amples values (A) for all food items and deriving a general per portion average across the broad range of all food items. The use of the serving unit Amples values (SUA) is particularly helpful for users of the present invention in monitoring consumed Amples (CA) when it comes to, for example, casseroles or other multi-ingredient dishes. Rather than going through the significant mental exercise of breaking down a food item into components, a user simply adds the appropriate number of Amples, as defined in FIG. 6, for the size of the portion consumed. The present invention thus provides a method for dieting that does not require the user to estimate weights of foods consumed, deconstruct food items, or count calories, carbohydrates or other units. To monitor his consumed Amples, a user must merely add the number of Amples for the portion consumed.

To calculate an Amples budget (AB), specifically the number of Amples that may be consumed per day, the present method requires the user to select a target weight (T) that he/she wants to achieve. Tables of ideal weight, published in the art including the Met Life Table, and certain governmental publications by the USDA and others, provide a useful guide but a user's body type and physiological characteristics must be factored in to select a reasonable, attainable target weight (T). The target weight (T) is then multiplied by a factor of nine (9) to arrive at the total permitted Amples per day, or Amples Budget (AB). The present invention requires that the user not surpass the calculated Amples Budget (AB) to achieve the desired target weight (T). Thus, a user can choose from any of the food they wish to eat. No specific foods are prohibited thus accounting for the cravings that dieters fight against.

Not only are no foods prohibited, but the participant can make small adjustments to the Amples Budget depending on their success. After a period, if the feelings of Satisfaction are not strong enough, the principles of the present invention dictate that the Amples Budget (AB) be adjusted by changing the Target Weight (T) in small increments. The inventors have discovered that if changes are small, the likelihood of failure is reduced. The principles of the present invention contemplate that the diet will be successful over a long period of time and that the diet will succeed by allowing participants to eat what they crave as long as the number of consumed Amples (CA) does not exceed the Amples Budget (AB).

If the user's goal is to maintain his/her current weight, the target weight (T) is defined as the current weight. In a significant aspect of the present invention, for user's seeking to lose weight by adopting a target weight lower than his/her current weight, the user is encouraged to choose an initial target weight higher than the ultimate, final target weight. The inventors have discovered that by attempting to lose in smaller amounts by first choosing more realistic target weights, the success rate is greater. This prevents the initial dramatic loss of weight that often leads to a later failure because the participants body changes were not ready for the dramatic changes.

While a preferred embodiment of the invention has been shown in the drawings and described, equivalent variations in the preferred embodiment may be apparent to those skilled in the art. However, the present invention should not be construed as limited to the specific form shown and described, but instead is as set forth in the following claims.

We claim:

1. A method for deriving an Amples value (A) for a food item comprising the steps of:
adopting the accepted kilocalorie value, E, for food item;
assigning a Passive Over Consumption value (P) to the food item, based on the propensity of said food item to elicit over consumption;
assigning a Satisfaction value (S) to the food item, based on the propensity of said food item ability to create the feeling of satisfaction;
calculating an Amples value for each said food item based on the formula, (E/P)/S.

2. The method set forth in claim 1 wherein said Passive Over Consumption Value (P) is an assigned constant ranging in value from 0.1 to 2.0.

3. The method set forth in claim 1 wherein said Satisfaction value (S) is an assigned constant ranging in value from 0.1 to 4.0.

4. The method set forth in claim 2 wherein said Satisfaction value (S) is an assigned constant ranging in value from 0.1 to 4.0.

5. A method for losing body weight by a user comprising the steps of:
defining an Amples Value (A) for every food item comprising the steps of:
adopting the accepted kilocalorie value (E) for said every food item;
assigning a Passive Over Consumption value (P) to for said every food item based on the propensity of said every food item to elicit over consumption;
assigning a Satisfaction value (S) for said every food item, based on the propensity of said every food item to create the feeling of satisfaction; and,
calculating an Amples value for said every food item based on the formula, A=(E/P)/S;
calculating a target weight (T) for said user;
calculating a daily Amples Budget (AB) by multiplying said target weight (T) by a factor of nine;
calculating Consumed Amples (CA) by adding said Amples values (A) for each food item consumed by said user per day to achieve a sum of Consumed Amples per day (CA);
maintaining a diet wherein said Consumed Amples (CA) is less than or equal to said Amples Budget (AB).

6. The method set forth in claim 5 further comprising the step of: modifying said target weight (T) according to the users feelings of lack of satisfaction and continual hunger in an amount that is less than their current weight.

7. The method set forth in claim 5 further comprising the step of: using a users current weight as the Target Weight, T.

8. The method set forth in claim 5 wherein the step of calculating Consumed Amples (CA) comprises the steps of:
assembling said Amples values (A) into a table;
creating said table on an easy to use form;
referencing said Amples values (A) from said table at the time food items referenced in said table are consumed;
summing said Amples values (A) for all items consumed.

9. A method for controlling body weight comprising the steps of:
calculating Amples values (A) for a large number of food items, comprising the steps of:
adopting the accepted kilocalorie values (E) for said food items;
assigning Passive Over Consumption values (P) to said food items, based on the propensity of said food items to elicit over consumption;
assigning Satisfaction values (S) to said food items, based on the propensity of said food items ability to create the feeling of satisfaction;
calculating Amples values (A) for said food items based on the formula, (E/P)/S.
assembling said Amples values (A) into a table;
averaging said Amples values (A) for said food items to arrive at a common representative factor;
using said common representative factor to calculate serving unit Amples values (SUA) for common serving units;
constructing food consumption guidelines utilizing common utensils and dishes.

10. The method of claim 9 wherein said common utensils are comprised of:
any flatware teaspoon;
any flatware tablespoon;
any flatware serving spoon;
any buffet serving spoon;
a dinner bowl;
a dinner plate; and,
a baking dish.
